(19) United States
(12) Patent Application Publication
(10) Pub. No.: US 2025/0268795 A1
(43) Pub. Date: Aug. 28, 2025

(54) METHOD FOR THAWING BIOLOGICALLY-DERIVED FROZEN MATERIAL BY VERY HIGH FREQUENCY ELECTROMAGNETIC WAVE IRRADIATION, CRYOPRECIPITATE PRODUCING METHOD, AND THAWING DEVICE

(71) Applicants: Hidenobu HARADA, Tokyo (JP); SMART HUNDRED CORPORATION, Miyagi (JP)

(72) Inventors: Minoru SATO, Miyagi (JP); Toshiyasu YAMAGUCHI, Miyagi (JP); Kazuhito SATO, Miyagi (JP); Naoyuki SAKUMA, Miyagi (JP); Satoshi KOSUNAGO, Miyagi (JP)

(21) Appl. No.: 18/851,407

(22) PCT Filed: Mar. 27, 2023
(86) PCT No.: PCT/JP2023/012195

§ 371 Date: Sep. 26, 2024

(30) Foreign Application Priority Data
Mar. 31, 2022 (JP) 2022-061438

(51) Int. Cl. A61J 3/00; A61K 35/16

(52) U.S. Cl. CPC A61J 3/00; A61K 35/16; A61J 2200/42

(57) ABSTRACT

A thawing method and a thawing apparatus for thawing a frozen substance of biological origin for medicine or research within a shorter time to reduce performance deterioration of the substance by avoiding contamination with germs and reducing temperature variations at thawing. High performance and safety are sought for thawed substances of biological origin particularly for medicine or research. The frozen substance of biological origin for medicine or research is irradiated with electromagnetic waves at 60 to 140 MHz to accelerate thawing and reduce performance deterioration of the frozen substance of biological origin.

### TECHNICAL FIELD

The present invention relates to a thawing method for a frozen substance of biological origin for medicine or research including irradiation with very-high-frequency electromagnetic waves particularly at 60 to 140 MHz, a method for producing cryoprecipitate, and a thawing apparatus.

### BACKGROUND OF ART

In medical, biological, physiological, and biochemical research, substances of biological origin frozen for storage or other purposes are thawed and widely used to analyze various biological reactions. Such substances of biological origin thawed over a long time or at too high thawing temperatures can be damaged and yield inaccurate experimental data. Currently, frozen substances of biological origin are typically thawed in thermostatic baths, refrigerators, or under running water, but can still be damaged. Methods for thawing frozen substances of biological origin in a shorter time with less damage are awaited.

High performance and safety are sought for thawed substances of biological origin particularly for medicine or research.

For example, a typical thawing method for frozen blood (referring to a frozen substance of blood origin, such as a frozen red blood cell solution or frozen blood plasma) includes immersing a frozen blood bag (a bag in which frozen blood is sealed) in a thermostatic bath containing warm water, as described in Patent Literature 1.

However, the thawing method with warm water immersion takes a long thawing time, as well as causing contamination with germs in warm water, performance deterioration of the thawed blood, and other usability and safety issues.

As described in Patent Literatures 2 and 3, known thawing methods may use no warm water and include vertically sandwiching a frozen blood bag between plates or bags containing heating-medium to warm the frozen blood bag using vertically installed heaters through, for example, the plates and swinging the structure including the vertically sandwiched frozen blood bag partially or entirely.

The components in the frozen blood bag are frozen at, for example, −30° C. or lower temperatures. The frozen blood bag in such a state can easily break at its periphery and can have concerns when sandwiched between, for example, the plates.

Patent Literature 4 describes a thawing method including suspending a frozen blood bag and blowing warm air from below. However, blowing warm air transfers heat to the frozen blood less efficiently than immersing frozen blood in warm water or sandwiching frozen blood with plates, taking a still longer thawing time.

With these known thawing methods, frozen blood is thawed by heating the surface of the frozen blood bag and conducting heat inward from outside the frozen blood. The methods can increase the temperature difference between the outer and inner portions of the frozen blood, adversely affecting the performances.

More specifically, frozen blood melted at temperatures exceeding 37° C. can have the coagulation factors being less active and solidifies at 50° C. or higher temperatures due to protein denaturation. For example, frozen blood having its inner portion with a temperature of −3° C. can have deteriorating performances when its outer portion has a temperature exceeding 37° C.

Cryoprecipitate is produced by slowly thawing frozen blood plasma at 4° C. for 24 to 30 hours. The production thus uses at least 2 days. To be ready for unexpected situations, methods for producing cryoprecipitate in a short time with less decrease in the collection rate of fibrinogen are awaited.

Human cells such as sperms or eggs have also been frozen for storage and used as appropriate. For increased childbirth at advanced maternal age and in vitro fertilization (IVF), sperms and eggs are to be safely stored, restored, and provided with acceleration.

### CITATION LIST

### Patent Literature

Patent Literature 1: Japanese Unexamined Patent Application Publication No. 2009-50316
Patent Literature 2: Japanese Unexamined Patent Application Publication No. 2013-252204
Patent Literature 3: Japanese Unexamined Patent Application Publication No. 2007-61245
Patent Literature 4: Japanese Unexamined Patent Application Publication No. 2001-218817

### SUMMARY OF INVENTION

### Technical Problem

Under such circumstances, one or more aspects of the present invention are directed to a thawing method and a thawing apparatus for thawing a frozen substance of biological origin within a shorter time to reduce performance deterioration of the substance by avoiding contamination with germs and reducing temperature variations at thawing.

### Solution to Problem

A thawing method for a frozen substance of biological origin for medicine or research according to one aspect of the present invention includes irradiating the frozen substance of biological origin with electromagnetic waves at 60 to 140 MHz to accelerate thawing and reduce performance deterioration of the frozen substance of biological origin.

In the thawing method according to the aspect of the present invention, the frozen substance of biological origin is frozen blood.

In the thawing method according to the aspect of the present invention, the frozen blood is a frozen red blood cell solution.

In the thawing method according to the aspect of the present invention, the frozen blood is frozen blood plasma.

A method for producing cryoprecipitate according to another aspect of the present invention uses the thawing method for frozen blood plasma.

In the thawing method according to the aspect of the present invention, the frozen substance of biological origin is frozen human cells.

A thawing apparatus for a frozen substance of biological origin for medicine or research according to another aspect of the present invention irradiates the frozen substance of biological origin with electromagnetic waves at 60 to 140 MHz to accelerate thawing and reduce performance deterioration of the frozen substance of biological origin.

### Advantageous Effects

As described above, the thawing method and the thawing apparatus according to the above aspects of the present invention can thaw a frozen substance of biological origin within a shorter time to reduce performance deterioration of the substance by avoiding contamination with germs and reducing temperature variations at thawing.

The method and the apparatus can promote, for example, reduction of discarded blood, prompt treatment for patients with rare blood types, contribution to disaster medicine, maintaining sanitary conditions, and use of autologous blood donations.

### BRIEF DESCRIPTION OF THE DRAWINGS

FIG. 1 is a graph showing temperature changes (temperature curves of thawing) at the center of a piece of a frozen substance of biological origin (5×5×4 cm; about 90 g) stored at −50° C. and thawed with electromagnetic waves at 60, 100, 140, 170, and 300 MHz.

FIG. 2 is a graph showing the time taken for the frozen substance of biological origin to reach 20° C. from −2° C. when the frozen substance is continuously thawed though irradiation with electromagnetic waves at 60 to 300 MHz.

FIG. 3 is a conceptual block diagram of a thawing apparatus for thawing a frozen substance of biological origin according to an embodiment of the present invention.

FIG. 4 is a front view of the thawing apparatus built based on the structure shown in FIG. 3.

FIG. 5 is a graph showing the comparison of the amounts of hemoglobin in a washing liquid.

FIG. 6 is a table showing measurement values of concentrations and activation of coagulation factors.

FIG. 7 is a graph showing example results of thawing a frozen human cell line.

### DETAILED DESCRIPTION

One or more embodiments of the present invention will now be described below, although the present invention is not limited to these embodiments.

A thawing method for a frozen substance of biological origin for medicine or research according to a first embodiment of the present invention includes irradiating the frozen substance of biological origin with electromagnetic waves at 60 to 140 MHz to accelerate thawing and reduce performance deterioration of the frozen substance of biological origin.

In the present embodiment, such very-high-frequency electromagnetic waves, or particularly, electromagnetic waves at 60 to 140 MHz, are highly permeable to the frozen substance of biological origin and can accelerate thawing of the frozen substance of biological origin with less temperature difference between the outer and inner portions of the substance of biological origin. The very-high-frequency electromagnetic waves refer to electromagnetic waves at 30 to 300 MHz inclusive.

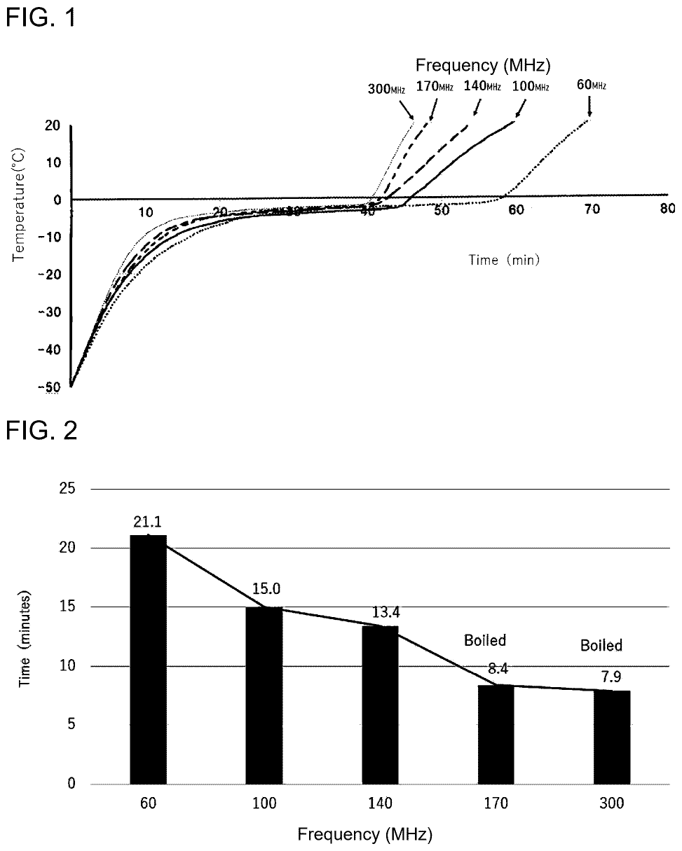

FIG. 1 is a graph showing temperature changes (temperature curves of thawing) at the center of a piece of a frozen substance of biological origin (5×5×4 cm; about 90 g) stored at −50° C., as an example frozen substance of biological origin for medicine or research, and thawed with electromagnetic waves at 60, 100, 140, 170, and 300 MHz.

FIG. 2 shows the measurement results of the time taken for the center of the piece of the frozen substance of biological origin to reach 20° C. from −2° C. when the frozen substance is thawed with electromagnetic waves at 60, 100, 140, 170, and 300 MHz.

The thawing with electromagnetic waves was performed with a thawing apparatus (described later). The output power of the electromagnetic waves was 25 W. The frequency and the output power of the electromagnetic waves were unchanged until the thawing was complete. The temperature was measured with a fiber-optic thermometer (manufactured by ASTECH) punctured into the center of the piece of the frozen substance of biological origin to the depth of 2.5 cm (2.5 cm from the surface).

A frozen substance of biological origin for medicine or research is to have higher performances and be safer than that for other uses (e.g., food).

Thawing with electromagnetic waves exceeding 140 MHz causes a sudden temperature increase after thawing, or at −2° C. or higher temperatures (refer to the results for 170 and 300 MHz in FIG. 1). The temperature is thus difficult to control and can easily exceed the target value (e.g., 37° C.), followed by tissue (cell) destruction or boiling at, for example, the edges of the frozen substance of biological origin (refer to the results for 170 and 300 MHz in FIG. 2). The thawing also causes non-uniform heat distribution in the frozen substance of biological origin, followed by various issues such as thaw drip and cell deterioration.

Although heating or other processes with electromagnetic waves at lower frequencies are typically less likely to affect thawing adversely and safer than heating with electromagnetic waves at higher frequencies, the thawing time lengthens suddenly at lower frequencies. In particular, with electromagnetic waves at a frequency below 60 MHz, thawing takes more than 70 minutes to heat the substance from −50 to 20° C. (refer to FIG. 2).

For the frozen substance to rapidly pass through the temperature range (−5 to −2° C.) in which various proteins (e.g., myoglobin and hemoglobin) in the substance of biological origin can easily denature (e.g., undergo metmyoglobin formation), electromagnetic waves at 60 MHz or higher frequencies may be used.

Based on the above conditions, a frequency bandwidth of 60 to 140 MHz is appropriate for thawing the frozen substance of biological origin for medicine or research.

A human origin substance as the frozen substance of biological origin for medicine or research is not readily available and difficult to experiment with. In the present embodiment, a frozen block of tuna is used to maintain high performances and safety. A frozen block of tuna is susceptible to temperature change, easy to experiment with (e.g., easy to determine the amount of thaw drip and the level of metmyoglobin formation and easy to partially measure the temperature of the substance that is not in the liquid form), and readily available.

The effects of frequency differences in electromagnetic wave irradiation on substances of biological origin differ in degree between humans and non-human animals (e.g., fish, cows, pigs, and chickens). However, both humans and non-human animals similarly have tissue including, for example, cells, various proteins, and water. Thus, no particular difference in effects is expected between humans and non-human animals.

More specifically, unlike known thawing methods of conducting heat inward from the surface, the method according to one or more embodiments of the present invention uses the principle of heat conversion, or more specifically, conversion of electromagnetic wave energy at 60 to 140 MHz to heat energy in the frozen substance of biological origin. With this method, heat can be conducted to the deepest portion of the substance uniformly across the outer and inner portions.

More specifically, electromagnetic waves at 60 to 140 MHz permeating through the frozen substance of biological origin vibrate water molecules in the frozen substance of biological origin. The vibrating water molecules generate intermolecular frictional heat to cause the frozen substance of biological origin to generate heat. The electromagnetic waves sufficiently permeating through the frozen substance of biological origin can heat the deep portion of the frozen substance of biological origin, as well as its surface. In other words, rather than conducting heat inward from the surface, the components of the frozen substance of biological origin generate heat. Thus, with the thawing method for the frozen substance of biological origin with the electromagnetic waves at 60 to 140 MHz according to one or more embodiments of the present invention, the frozen substance of biological origin can be efficiently heated across the components.

When the thawing method based on the known principle uses electromagnetic waves other than the very-high-frequency electromagnetic waves at 60 to 140 MHz, temperature differences occur between the inner and outer portions of the frozen substance of biological origin.

In other words, when the electromagnetic waves are used from the frozen state of the substance until the substance is thawed, the temperature of the outer portion may increase and cause performance deterioration although the inner portion remains frozen. To avoid performance deterioration, for example, a process of applying electromagnetic waves for 15 seconds and pausing for 30 seconds to cool the outer portion may be repeated. However, continuous irradiation with electromagnetic waves is difficult.

The method according to one or more embodiments of the present invention does not conduct heat from the surface, and can perform continuous irradiation from the frozen state of the substance until the substance is thawed without pausing.

This allows acceleration of thawing with high energy efficiency.

Continuous irradiation may be performed at the same frequency using the same irradiation apparatus.

Compared with a known thawing apparatus using warm water, the thawing apparatus according to one or more embodiments can be immediately used after turning on the switch without setting and maintaining the thawing temperature. The apparatus can be promptly ready for unexpected situations with less energy consumption.

In one or more embodiments of the present invention, the frozen substance of biological origin for medicine or research is, for example, a frozen liquid (of biological origin) or a frozen solid containing a substance of biological origin. More specifically, examples of the frozen substance of biological origin include cell suspensions and blood. Examples of the cell suspensions include hematopoietic stem cells (e.g., umbilical cord blood, bone marrow fluid, and peripheral blood stem cells) for stem cell transplantation. Cells of the cell suspensions include, but are not limited to, adherent cells such as myoblasts, cardiomyocytes, fibroblasts, synovial cells, epithelial cells, endothelial cells, hepatocytes, pancreatic cells, renal cells, adrenal cells, periodontal ligament cells, gingival cells, periosteal cells, skin cells, or cartilage cells, blood cells or blood components such as whole blood, red blood cells, white blood cells, lymphocytes (T lymphocytes and B lymphocytes), dendritic cells, blood plasma, platelets or platelet-rich plasma, or cells such as bone mononuclear cells, hematopoietic stem cells, embryonic stem (ES) cells, pluripotent stem cells, induced pluripotent stem (iPS) cell-derived cells (e.g., iPS cell-derived cardiomyocytes), mesenchymal stem cells (e.g., cells derived from bone marrow, adipose tissue, peripheral blood, skin, hair roots, muscle tissue, endometrium, placentas, or umbilical cord blood), sperm cells, or egg cells. These cells may be cells to which genes have been introduced for use in, for example, gene therapy.

Any frozen substance of biological origin for medicine or research may be included in the frozen substance of biological origin according to one or more embodiments of the present invention.

Embodiments of the present invention may be directed to frozen substances of biological origin in humans as well as frozen substances of biological origin in non-human animals.

The terms thawing and melting herein have the same meaning.

Similarly, frozen (Toketsu in Japanese) and frozen (Reito in Japanese) have the same meaning.

The frozen substance of biological origin may be stored in a container for sanitation.

The container may be any container capable of wrapping the frozen substance of biological origin, including plastic wrapping, in one or more embodiments of the present invention.

The container may be formed from materials that absorb a smaller amount of very-high-frequency electromagnetic waves than the frozen substance of biological origin.

The container may be formed from, for example, a resin material (e.g., a soft bag formed from an easily deformable resin film) or a hard material.

Typically, a resin material absorbs a smaller amount of very-high-frequency electromagnetic waves than a frozen substance of biological origin. Thus, a container formed from a resin material reduces temperature increase or degeneration of the frozen substance of biological origin resulting from the electromagnetic waves absorbed by the container. The container may generally be flat, and may be rectangular, circular, or oval in a plan view.

In a second embodiment of the present invention, frozen blood is used as the frozen substance of biological origin in the thawing method for a frozen substance of biological origin according to the first embodiment.

Blood contains cellular components (blood cells) such as red blood cells, white blood cells, and platelets, and liquid components (blood plasma). The ratio of the cellular components to the blood plasma components is approximately 45 to 55.

Blood plasma is a solution mainly containing various proteins such as fibrinogen as a protein associated particularly with blood coagulation.

In the present embodiment, when the frozen substance of biological origin is frozen blood, the thawed substance is a liquid containing salt. Thus, electromagnetic waves at a frequency in the range of 60 to 140 MHz accelerates thawing, avoiding denaturation of proteins, burning, and boiling.

To avoid coagulation resulting from protein denaturation, the temperature may constantly be below 50° C. To avoid decreased activation of the coagulation factors, the temperature may be 37° C. or lower.

In one or more embodiments of the present invention, the frozen blood is, but not limited to, a frozen red blood cell solution or frozen blood plasma as described above. Frozen blood in one or more embodiments of the present invention may be any frozen substance of blood origin.

For frozen blood, more appropriate frequencies of the electromagnetic waves may be 65 MHz or higher frequencies for accelerated thawing. The electromagnetic waves at 135 MHz or lower frequencies may be used to further reduce performance deterioration.

In a third embodiment of the present invention, a frozen red blood cell solution is used as the frozen blood in the thawing method for a frozen substance of biological origin according to the second embodiment.

Frozen red blood cell solutions mainly used for blood transfusions are stored at 2 to 6° C. after thawing and have a storage period of 4 days after production. Thus, unused blood is discarded.

In the present embodiment, appropriate thawing is performed to lower the hemolytic rate during washing of the thawed red blood cell solution, compared with known thawing methods. This reduces extra thawing of frozen red blood solutions and discarded blood.

Frozen red blood cell solutions are less likely to have deterioration of the red blood cells with lower frequencies of the electromagnetic waves. Thus, the upper limit value of the frequency range (60 to 140 MHz) of the electromagnetic waves described above may be 130 or 125 MHz, or 120 or 115 MHz. For accelerated thawing, the lower limit value of the frequency range of the electromagnetic waves described above may be 70 or 75 MHz, or 80 or 85 MHz.

In a fourth embodiment of the present invention, frozen blood plasma is used as the frozen blood in the thawing method for a frozen substance of biological origin according to the second embodiment.

Known methods for thawing frozen blood plasma use warm water at 30 to 37° C. for melting and take time, causing sanitary concerns such as contamination with disease-causing germs.

The method according to the present embodiment accelerates thawing without such sanitary concerns while reducing performance deterioration.

Frozen blood plasma thawed with acceleration is less likely to have protein deterioration. Thus, the lower limit value of the frequency range (60 to 140 MHz) of the electromagnetic waves described above may be 70 or 75 MHz, or 80 or 85 MHz. To further reduce the overall performance deterioration of the frozen blood plasma, the upper limit value of the frequency range of the electromagnetic waves described above may be 130 or 125 MHz, or 120 or 115 MHz.

A method for producing cryoprecipitate according to a fifth embodiment of the present invention includes the thawing method according to the fourth embodiment.

Frozen blood plasma that has been administered as hemostatic therapy contains coagulation factors such as fibrinogen at a normal concentration. Thus, a large amount of blood plasma is administered for rapid fibrinogen replenishment in severe bleeding, possibly placing a burden on the patient. In response to this concern, cryoprecipitate, including concentrated coagulation factors such as fibrinogen, is prepared and used in hospitals. However, the production involves longer thawing time and other issues.

The method according to the present embodiment accelerates production of cryoprecipitate without reducing the collection rate of fibrinogen.

The method for producing (preparing) cryoprecipitate according to one or more embodiments of the present invention uses, among other known methods, the thawing method according to one or more embodiments of the present invention in the process of thawing (melting) the frozen blood plasma. Processes other than thawing (melting) may be performed with known methods.

Examples of known methods for producing (preparing) cryoprecipitate include Melting Method of Fresh Frozen Plasma (FFP) published on the website of the Japanese Red Cross Society and Protocol for Production of Cryoprecipitate published on the website of The Japan Society of Transfusion Medicine and Cell Therapy.

Cryoprecipitate thawed with acceleration can have less protein deterioration. Thus, the lower limit value of the frequency range (60 to 140 MHz) of the electromagnetic waves described above may be 70 or 75 MHz, or 80 or 85 MHz. To further reduce the overall performance deterioration, the upper limit value of the frequency range of the electromagnetic waves described above may be 130 or 125 MHz, or 120 or 115 MHz.

In a sixth embodiment of the present invention, frozen human cells are used as the frozen substance of biological origin in the thawing method for a frozen substance of biological origin according to the first embodiment.

For frozen human cells, electromagnetic waves at lower frequencies cause less deterioration of the human cells. Thus, the upper limit value of the frequency range (60 to 140 MHz) of the electromagnetic waves described above may be 130 or 125 MHz, or 120 or 115 MHz. For accelerated thawing, the lower limit value of the frequency range of the electromagnetic waves described above may be 70 or 75 MHz, or 80 or 85 MHz.

The method according to the present embodiment accelerates appropriate thawing of frozen human cells and reduces performance deterioration.

A thawing apparatus for a frozen substance of biological origin for medicine or research according to a seventh embodiment of the present invention irradiates the frozen substance of biological origin with electromagnetic waves at 60 to 140 MHz to accelerate thawing and reduce performance deterioration of the frozen substance of biological origin.

In the present embodiment, the thawing apparatus is used to produce the effects described in the first to sixth embodiments of the present invention on the frozen substance of biological origin for medicine or research.

The thawing apparatus will be described in detail below, followed by examples performed with the thawing apparatus.

### Thawing Apparatus

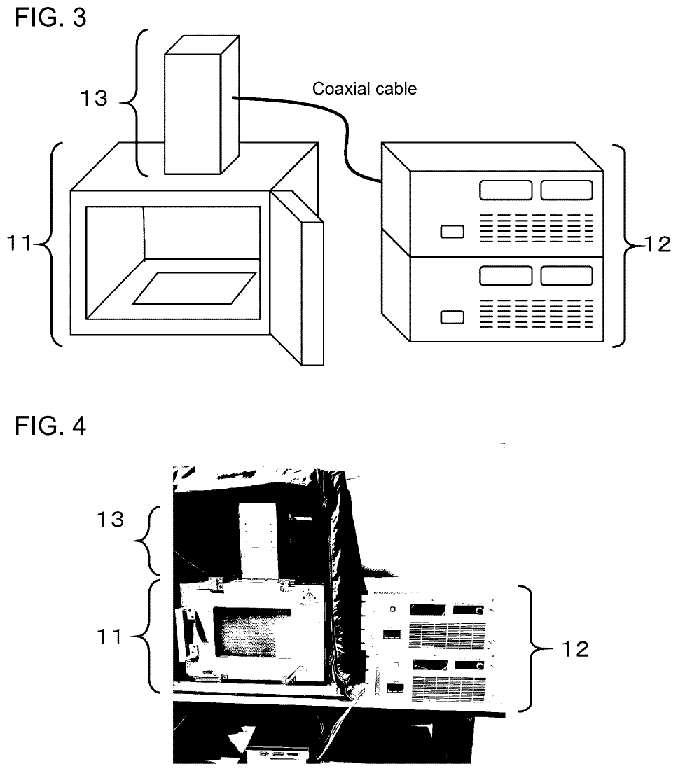

FIG. 3 is a conceptual block diagram of the thawing apparatus according to one or more embodiments of the present invention. The thawing apparatus includes an irradiation cavity 11, an amplifier 12, and a matcher 13. The irradiation cavity 11 contains an antenna. The matcher 13 detects the intensity of the applied electromagnetic waves and the intensity of the reflected electromagnetic waves to use the difference between the two intensities as the actual output (W), which is adjusted to the initial set value. The matcher 13 is one of multiple matchers each corresponding to a different frequency band, and is replaceable for the frequency band being used.

FIG. 4 shows the thawing apparatus built based on the structure shown in FIG. 3. The reference signs in FIG. 4 correspond to the reference signs in FIG. 3.

### REFERENCE SIGNS LIST

11 irradiation cavity
12 amplifier
13 matcher

The thawing method for a frozen substance of biological origin for medicine or research according to one or more embodiments of the present invention will be described in detail below with examples. The frozen substance of biological origin in each example is human origin and stored in a container. However, the substance may not be stored in a container and thawed in the same manner as when stored in a container.

As the electromagnetic waves at 60 to 140 MHz to be applied, electromagnetic waves at 100 MHz (or 100±10 MHz including irradiation error), which is a middle value, are used.

First to third examples of the present invention will be described below, although the present invention is not limited to these examples.

### First Example

### Thawing of Frozen Red Blood Cell Solution

Frozen red blood cell solutions for medicine or research are to be thawed with acceleration without having hemolysis promotion.

In the first example, the hemolytic rate during washing was measured for red blood cells thawed with a known method using warm water and red blood cells thawed through irradiation with the electromagnetic waves described above.

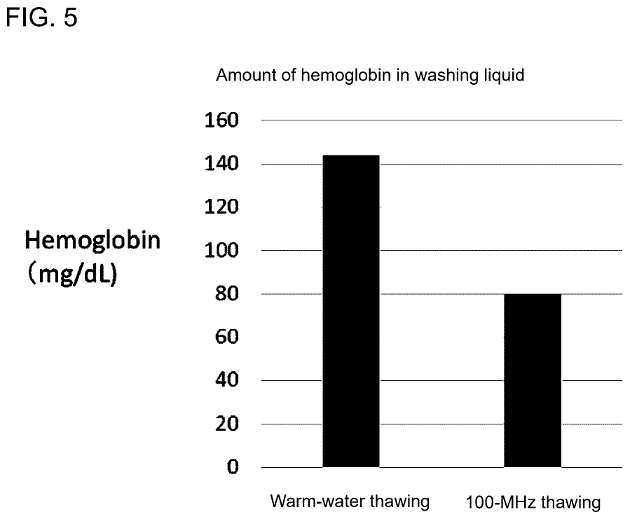

The measurement results are shown in FIG. 5. The amounts of hemoglobin in the figure refer to the amounts of the red pigment representing hemoglobin contained in the washing liquid and derived from the red blood cells hemolyzed during washing for removing cryoprotectant (e.g., glycerol), which is added to prevent hemolysis when freezing the red blood cells. Thus, a smaller amount of hemoglobin, indicating a lower degree of hemolysis, is appropriate.

More specifically, the first example demonstrates, as shown in FIG. 5, that the amount of hemoglobin in the washing liquid is smaller with the thawing method including irradiation with the electromagnetic waves described above than with the known method, indicating that the present thawing method is more appropriate.

### Second Example

### Preparation of Cryoprecipitate

Typically, cryoprecipitate is produced by separating fresh frozen blood plasma (FFP) slowly melted (thawed) at 1 to 6° C. into a precipitate and a supernatant and removing the supernatant by centrifugation. Cryoprecipitate mostly contains fibrinogen, coagulation factor VIII, von Willebrand factor (VWF), coagulation factor XIII, fibronectin, and platelet microparticles. The precipitated fraction is resuspended in a small amount of blood plasma and cryopreserved to produce cryoprecipitate, which is melted (thawed) as appropriate in use.

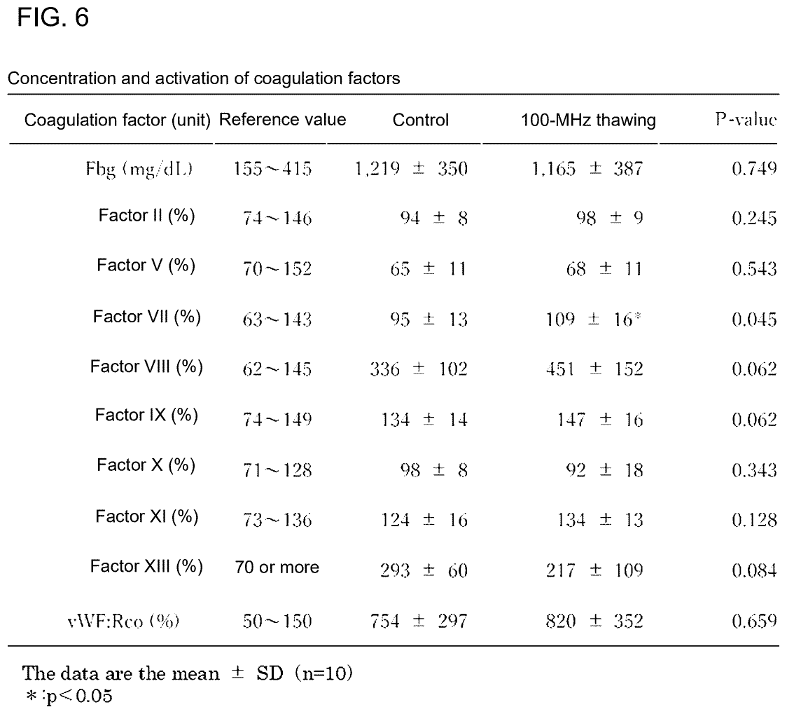

In the second example, as shown in FIG. 6, fresh frozen blood plasma is irradiated with the electromagnetic waves described above in the thawing process to produce cryoprecipitate.

The prepared cryoprecipitate was thawed within a shorter time (25 minutes) than with a known thawing method (at 4° C. for 20 hours) and remained active.

### Third Example

### Thawing of Frozen Human Cell Line

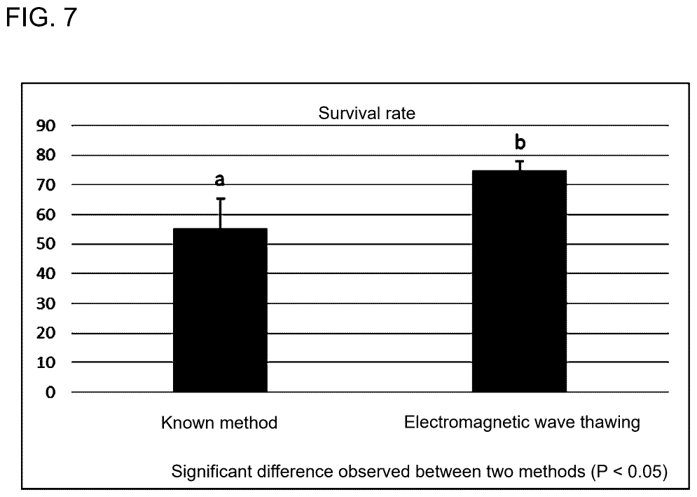

In the third example, a human cell line (HL60) was subcultured and grown in RPMI 1640 culture medium (10% fetal bovine serum or FBS) and stored in a frozen culture medium (−80° C.). FIG. 7 shows survival rate comparison of the human cell line between when the frozen cells were thawed through irradiation with the electromagnetic waves described above for 2 minutes (electromagnetic wave thawing) and when the frozen cells were thawed in warm water at 37° C. (known method).

The survival rates were calculated by measuring the absorbance at 450 nm after seeding 100 μl of the culture medium at 5×104 cells/ml concentration into a 96-well plate, adding Cell Counting Kit-8 (DOJINDO LABORATORIES), and then leaving the plate in a CO2 incubator for 150 minutes.
(https://www.dojindo.co.jp/manual/CK04.pdf).

As shown in FIG. 7, the survival rate is higher for the electromagnetic wave thawing than for the known method. Thus, the thawing method described above is appropriate and causes less damage to the cells than the known method.

1. A method for producing cryoprecipitate, the method comprising:
irradiating a frozen blood plasma with electromagnetic waves at 60 to 140 MHz to accelerate thawing of the frozen blood plasma and reduce performance deterioration of the frozen blood plasma; and
separating the thawed blood plasma into a precipitate and a supernatant and removing the supernatant.

2. A method for producing cryoprecipitate, the method comprising:
irradiating a frozen blood plasma with electromagnetic waves at 60 to 130 MHz to accelerate thawing of the frozen blood plasma and reduce performance deterioration of the frozen blood plasma; and
separating the thawed blood plasma into a precipitate and a supernatant and removing the supernatant.

3-7. (canceled)
